(19) United States
(12) Patent Application Publication
(10) Pub. No.: US 2022/0354456 A1
(43) Pub. Date: Nov. 10, 2022

(54) ACOUSTIC COUPLER, ULTRASOUND IMAGE PROCESSING METHOD, AND ULTRASOUND IMAGING DEVICE

(71) Applicant: FUJIFILM Healthcare Corporation, Kashiwa-shi (JP)

(72) Inventors: Kenichi Kawabata, Chiba (JP); Hirozumi Takeshima, Chiba (JP)

(21) Appl. No.: 17/729,139

(22) Filed: Apr. 26, 2022

(30) Foreign Application Priority Data
May 6, 2021 (JP) 2021-078765

(51) Int. Cl. A61B 8/00; G01N 29/28

(52) U.S. Cl. CPC A61B 8/4281; G01N 29/28; G01N 29/26

(57) ABSTRACT

Provided is an acoustic coupler for ultrasound imaging that can detect a posture of an ultrasound probe from an image. An acoustic coupler includes: a first layer in contact with an ultrasound probe of an ultrasound imaging device; a second layer in contact with an imaging target; and an intermediate layer made of a material having a high elastic modulus between the first layer and the second layer. The intermediate layer includes a plurality of markers made of a material that reflects an ultrasound wave, at different positions in an ultrasound wave propagation direction, for example, near a boundary between two adjacent layers. The ultrasound imaging device detects the posture of the ultrasound probe based on images of the markers included in an ultrasound image.

### BACKGROUND OF THE INVENTION

### 1. Field of the Invention

The present invention relates to an acoustic coupler interposed between an ultrasound probe and an inspection target in an ultrasound imaging device such as a medical ultrasound diagnostic device.

### 2. Description of the Related Art

In modern medical care, image diagnosis in which information in a body is obtained non-invasively is an essential technique and is widely used. In an image diagnosis modality, there are great expectations for an ultrasound diagnostic device that can provide small and inexpensive solutions.

In particular, there is a high need for a simple ultrasound diagnostic device for screening tests such as pneumonia. However, there is a problem of dependence on an operator that since body hair and pores are present on a body surface of a subject, a quality of an image quality depends on an angle and a method of scanning when an ultrasound probe (hereinafter, referred to as a probe) is pressed against the body surface, a thickness of jelly applied to the body surface of the subject, and the like, and a high image quality cannot be obtained unless the operator is an expert. An acoustic coupler using a high-deformation gel instead of the jelly has also been developed. By using the high-deformation gel, the problem of dependence on the operator who applies the jelly can be reduced, and a work involved in application and removal can be reduced.

When the acoustic coupler is used, it is necessary to optimize an acoustic impedance in order to reduce an attenuation of ultrasound waves between an acoustic lens or the like arranged on a contact surface side of the probe and the subject. JP-A-05-27784 discloses that an acoustic coupler is formed by laminating materials having different acoustic impedances.

However, when the acoustic coupler is used in the ultrasound diagnostic device, the acoustic coupler is required to be able to be deformed following unevenness of a surface of an inspection target and to be excellent in acoustic matching with the subject, but it is difficult for the acoustic couplers in the related art to satisfy both requirements, and the acoustic couplers in the related art are hardly used in a clinical field.

To solve the problem, the present applicant has developed and proposed a resin for an acoustic coupler having an excellent acoustic characteristic and high deformability (JP-A-2021-10571, or the like). By using such a resin having a high deformability, it is possible to perform imaging by following an uneven shape without distorting the surface of the subject.

### SUMMARY OF THE INVENTION

When ultrasound imaging is performed for a purpose of screening, it is important to capture an image of a target site without omission. However, due to the unevenness of the body surface, for example, when scanning is performed while moving the probe, there is a possibility that a direction of an ultrasound beam changes, a gap is generated between scans, and therefore the target site cannot be covered. It is difficult to confirm the gap generated between such scans by comparing images of the scans. Therefore, even if there is a gap that cannot be scanned between adjacent imaging surfaces, the gap cannot be confirmed, and as a result, there is a possibility that omission occurs in the imaging of the target site.

An object of the invention is to provide a means capable of capturing an image of a target site without omission, and more specifically, to provide an acoustic coupler capable of grasping a positional relationship between images based on a plurality of images obtained by ultrasound imaging.

The invention solves the above problem by embedding markers made of a material that reflects an ultrasound wave in a resin (gel) forming the acoustic coupler. By arranging the markers at two or more different positions with respect to an ultrasound wave propagation direction, an imaging surface determined by the ultrasound wave propagation direction can be reliably specified.

That is, an acoustic coupler of the invention includes: a first layer in contact with an ultrasound probe (hereinafter, it is also simply referred to as a probe) of an ultrasound imaging device; and a second layer in contact with an imaging target. A plurality of markers are provided between the first layer and the second layer at different positions with respect to an ultrasound wave propagation direction. For example, an intermediate layer having a high elastic modulus is arranged between the first layer and the second layer, and the markers are arranged above and below the intermediate layer.

An ultrasound image processing method of the invention is for arranging an acoustic coupler between an inspection target and an ultrasound probe, transmitting and receiving an ultrasound wave to and from the inspection target via the ultrasound probe, and processing a generated ultrasound image of the inspection target. The ultrasound image processing method includes: specifying a plurality of markers of the acoustic coupler in the ultrasound image using the acoustic coupler of the invention as the acoustic coupler; and calculating position information of the ultrasound probe using a positional relationship of the plurality of specified markers.

An ultrasound imaging device of the invention includes: a transmission and reception unit to which an ultrasound probe is connected and configured to transmit and receive an ultrasound wave via the ultrasound probe; an image generation unit configured to generate an ultrasound image using an ultrasound wave that is a reflected wave received from an inspection target; and an imaging position calculation unit configured to calculate position information of the ultrasound probe based on a position of an image of a marker included in the ultrasound image. The ultrasound image is an image captured by interposing the acoustic coupler of the invention between the inspection target and the ultrasound probe.

According to the invention, by embedding the markers in the acoustic coupler, a position and a scanning direction of the probe can be confirmed based on the positions of the markers included in the ultrasound image captured via the acoustic coupler. Therefore, a position of a surface (that is, the imaging surface) scanned by the probe can be confirmed.

### BRIEF DESCRIPTION OF THE DRAWINGS

FIG. 1 is a cross-sectional view showing an acoustic coupler according to an embodiment of the invention.

FIGS. 2A and 2B are diagrams showing types and arrangement examples of markers, respectively.

FIG. 3 is a diagram showing an example of a method for manufacturing an acoustic coupler of the invention.

FIG. 4 is a diagram showing another example of the method for manufacturing the acoustic coupler of the invention.

FIG. 5 is a diagram showing an example of a usage state of the acoustic coupler of the invention.

FIGS. 6A and 6B are diagrams illustrating a case where a long axis direction of an ultrasound probe is parallel to the arrangement of the markers of the acoustic coupler, in which FIG. 6A is a diagram showing a relationship between the markers and the long axis direction, and FIG. 6B is a diagram showing images (point images) of the markers in an ultrasound image when the ultrasound probe is at a position of FIG. 6A.

FIGS. 7A and 7B are diagrams illustrating images when the markers are present on only one surface.

FIGS. 8A and 8B are diagrams illustrating images when the markers are present on two surfaces.

FIGS. 9A and 9B are diagrams showing a case where the long axis direction of the ultrasound probe is inclined at an angle θ with respect to the markers of the acoustic coupler, in which FIG. 9A is a diagram showing a relationship between the markers and the long axis direction, and FIG. 9B is a diagram showing images (point images) of the markers in the ultrasound image when the ultrasound probe is at a position of FIG. 9A.

FIGS. 10A and 10B are diagrams illustrating a method for distinguishing a warp and a weft from a mesh-shaped marker.

FIGS. 11A and 11B are diagrams illustrating a detection method when the ultrasound probe moves along the long axis direction, in which FIG. 11A is a diagram showing a relationship between the markers and the long axis direction, and FIG. 11B is a diagram showing a change in the images (point images) of the markers before and after the ultrasound probe is moved.

FIGS. 12A and 12B are diagrams illustrating a detection method when the ultrasound probe moves along a short axis direction, in which FIG. 12A is a diagram showing a relationship between the markers and the long axis direction, and FIG. 12B is a diagram showing a change in the images (point images) of the markers before and after the ultrasound probe is moved.

FIGS. 13A and 13B are diagrams illustrating a detection method when an imaging surface is inclined with respect to a reference surface, in which FIG. 13A shows a case where the imaging surface is perpendicular to the reference surface, and FIG. 13B shows a case where the imaging surface is inclined with respect to the reference surface.

FIG. 14 is an overall view showing an ultrasound imaging device according to an embodiment of the invention.

FIG. 15 is a flowchart showing an example of an operation of an imaging position calculation unit.

FIG. 16 is a diagram showing a display example of position information calculated by the imaging position calculation unit.

### DESCRIPTION OF THE PREFERRED EMBODIMENTS

Hereinafter, an embodiment of an acoustic coupler of the invention and an embodiment of an ultrasound imaging method using the acoustic coupler will be described.

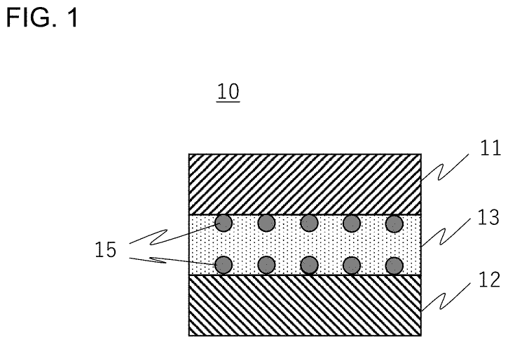

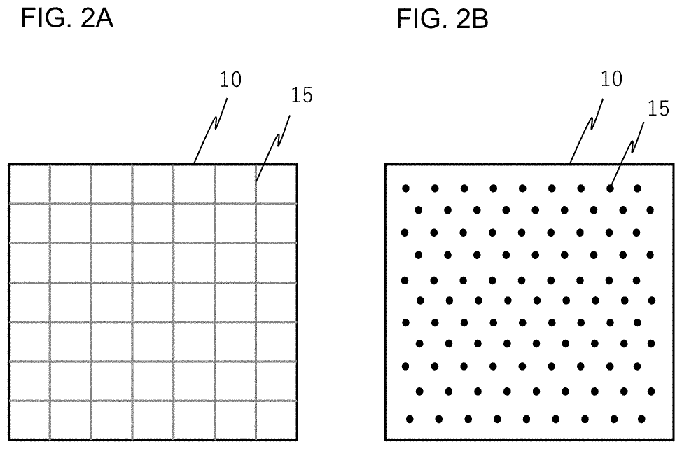

First, the embodiment of the acoustic coupler will be described with reference to FIGS. 1 and 2.

An acoustic coupler 10 according to the present embodiment is formed by a plurality of layers each made of a polymer gel, and markers identifiable in an ultrasound image are arranged between the layers or inside the layers. For example, as shown in FIG. 1, the acoustic coupler 10 includes a layer (first layer) 11 that intervenes with an ultrasound imaging device (ultrasound probe), a layer (second layer) 12 that intervenes with an imaging target, and an intermediate layer 13 that intervenes between the first layer 11 and the second layer 12. In the example shown in FIG. 1, a plurality of markers 15 are held and fixed at two places of the intermediate layer 13 near a boundary with the first layer 11 and near a boundary with the second layer 12.

Although FIG. 1 shows the acoustic coupler having three layers, the acoustic coupler may have a multilayer structure in which another layer is inserted between these layers or inside the layers, and the markers 15 may be arranged at any position as long as the markers 15 are arranged between the first layer 11 and the second layer 12.

It is desirable that the polymer gel, which is a material of each layer of the acoustic coupler 10, can achieve both an acoustic characteristic and a mechanical characteristic required for ultrasound imaging. Regarding the acoustic characteristic, for example, it is desirable that a sound velocity value is equivalent to a sound velocity value of water (within deviation of 5%) and an ultrasound attenuation rate is 0.1 dB/MHz/cm or less. The mechanical characteristic differs depending on a function of the layer, and it is preferable that the layer (second layer) 12 that intervenes an imaging target 30 has a high deformability, and an intermediate layer holding the markers 15 has a high elastic modulus.

Specifically, the material of the polymer gel can use hydrogel made of cross-linked polymers such as agarose (agar), acrylamide, polysaccharide or a mixture of acrylamide and polysaccharide and water, and a non-hydrogel containing organic solvents such as alcohol. In particular, the hydrogel using acrylamides such as (meth) acrylamide, N-methyl (meth) acrylamide, N-propyl (meth) acrylamide, N, N-dimethyl (meth) acrylamide, N, N-diethyl (meth) acrylamide, diacetone acrylamide, N-hydroxydiethylacrylamide, N-(3-methoxypropyl) acrylamide, and N-isopropylacrylamide as polymerizable monomers is preferable because the hydrogel has the acoustic characteristic close to that of water and can reach a deep portion without attenuating ultrasound waves. When the acrylamide is used as the polymerizable monomer, it is preferable to use (bis) acrylamide such as N, N′-methylene (bis) acrylamide and N, N′-ethylene (bis) acrylamide as a cross-linking agent.

Further, the second layer 12 that intervenes with the imaging target contains polyacrylamide having a network structure and alginic acid that are disclosed in JP-A-2021-10571, and it is preferable to use the hydrogel having a structure in which the alginic acid is held in a network having a network structure of the polyacrylamide. This gel has a small elastic modulus (10 kPa or less) and high deformability (100% or more) required for ultrasound measurement, and can be deformed following unevenness of a surface of a subject.

The materials of the first layer 11, the intermediate layer 13, and the second layer 12 may be different from each other, but by using the same type of gel material, a bondability between the layers can be enhanced. Even when the same type of gel material is used, an acoustic impedance of each layer may be adjusted by changing gelation of the gel constituting each layer, a ratio of water, and the like, adding an additive such as fine particles to the layer, or adjusting an amount of the gel material, in order to impart a gradient to the acoustic impedance or to cope with other purposes of use. By adjusting a composition of the layer, it is possible to adjust not only the acoustic impedance but also a physical property such that, for example, a side to be brought into contact with the probe is slippery and a side to be brought into contact with a living body has adhesiveness.

The intermediate layer 13 is a layer for fixing the markers 15 so as not to move in the gel, and it is preferable that an elastic modulus of the intermediate layer 13 is higher than the elastic moduli of the first layer 11 and the second layer 12. Specifically, it is preferable that the elastic modulus of the intermediate layer 13 is about 10 kPa or more in Young's modulus, and the elastic modulus may be appropriately adjusted to 10 kPa or more for a robot and about 10 kPa by a hand, depending on whether a probe operation is performed by the robot (for the robot) or the hand. In any case, by setting the elastic modulus of the intermediate layer 13 to be high, for example, even when the probe is pressed, deformation of the intermediate layer 13 is suppressed, and the markers held in the intermediate layer 13 can function as indexes of positions in an ultrasound scanning direction without moving.

The intermediate layer 13 having such an elastic modulus can also be formed by a gel having a material different from those of the first layer 11 and the second layer 12, but is preferably formed by the same type of material in order to enhance the bondability between the layers, and in this case, the elastic modulus of the intermediate layer 13 can be adjusted by adjusting a concentration of the polymerizable monomer, a ratio of the polymerizable monomer to the cross-linking agent, an additive added at a time of gelation, and the like. For example, in the case of the gel using the acrylamide as the polymerizable monomer described above and the (bis) acrylamide as the cross-linking agent, the elastic modulus (Young's modulus) can be increased by increasing a concentration of one or both of the acrylamide and the (bis) acrylamide.

The markers 15 can be made of, for example, a material that reflects an ultrasound wave, such as a striatum (wire), particles, and bubbles. Specific examples of the material that reflects the ultrasound wave include a metal, a metal oxide, glass, ceramics, and a gas such as air as long as the acoustic impedance is significantly different from an acoustic impedance of the imaging target, and the material can be appropriately selected according to a form of the markers 15. By arranging the material that reflects the ultrasound wave at a position closer to the ultrasound probe than the imaging target, a reflected signal stronger than the ultrasound wave reflected from the imaging target is received by the ultrasound imaging device, and positions of the markers can be grasped on the ultrasound image. By detecting the positions of the markers, an angle and inclination of the ultrasound probe can be detected, and an imaging surface can be specified. A method for detecting a position of the ultrasound probe will be described later.

As shown in FIGS. 2A and 2B, the markers 15 are arranged in a two-dimensional direction with respect to a plane direction of the acoustic coupler. FIG. 2A is an example of a wire, and FIG. 2B is an example of particles or bubbles. FIG. 2 shows an arrangement in one plane, but as shown in FIG. 1, the markers 15 are arranged at different positions (different surfaces) in an irradiation direction of the ultrasound waves. The markers 15 may be arranged in the same manner on different surfaces, but can also be arranged in different manners on different surfaces, and therefore, in a position detection of the ultrasound probe to be described later, a movement amount of the ultrasound probe can be detected regardless of the angle of the ultrasound probe with respect to an arrangement direction of the markers. FIG. 2 shows a case where an upper surface shape of the acoustic coupler 10 is rectangular or square, but the shape of the acoustic coupler 10 is not limited to this, and may be any shape such as a circle or an ellipse.

A surface (layer) on which the markers 15 are arranged may be inside the intermediate layer 13 as shown in FIG. 1, or may be a boundary surface between the intermediate layer 13 and the first layer 11 and a boundary surface between the intermediate layer 13 and the second layer 12. However, in either case, even if the first layer 11 and the second layer 12 are deformed, the positions of the markers 15 with respect to the intermediate layer 13 are fixed. Therefore, even if the first layer 11 or the second layer 12 is deformed, a positional relationship between the probe and the marker is maintained.

A thickness of each layer constituting the acoustic coupler 10 and a size of the marker are not particularly limited, but a thickness of the entire acoustic coupler 10 is preferably about 5 mm to 20 mm in order to minimize the attenuation of the ultrasound waves by the acoustic coupler as much as possible, and to ensure an imaging region of the subject as much as possible while absorbing the unevenness of the subject by the second layer 12. When a measurement is automatically performed by a robot or the like, an optimum value changes according to a configuration of a measurement system. The thickness of each layer may be the same or different, but it is preferable that the second layer has a thickness at least capable of absorbing the unevenness of the subject.

The size of the marker may be any size as long as the marker can be detected as a point image or a line image on an image, and a line width is preferably about 0.1 mm to 5 mm if the marker is the striatum. A particle diameter of bubbles or particles is preferably about 0.01 mm to 0.1 mm.

The acoustic coupler configured as described above can be manufactured by, for example, the following manufacturing method.

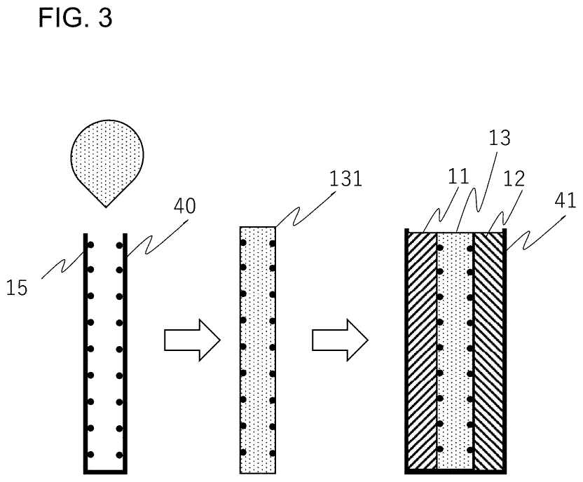

When the markers 15 are arranged in the intermediate layer 13, as shown in FIG. 3, first, markers (for example, wire meshes) are arranged in a mold 40 for molding intermediate layer, and a gel or a resin composition constituting the intermediate layer is put into the mold and cured by a chemical or physical operation to produce an intermediate layer sheet 131 in which the markers 15 are arranged in two places (two layers) inside.

The intermediate layer sheet 131 is vertically arranged in another molding mold 41, a gel or a resin composition of a first layer and a gel or a resin composition of a second layer are injected into both sides of the intermediate layer sheet 131, both the compositions are cured by the chemical or physical operation to be gelled, and the acoustic coupler 10 in which the first layer 11 is formed on one side surface of the intermediate layer 13 and the second layer 12 is formed on another side surface of the intermediate layer 13 is manufactured. In this method, the acoustic coupler in which the markers are arranged inside the intermediate layer can be manufactured.

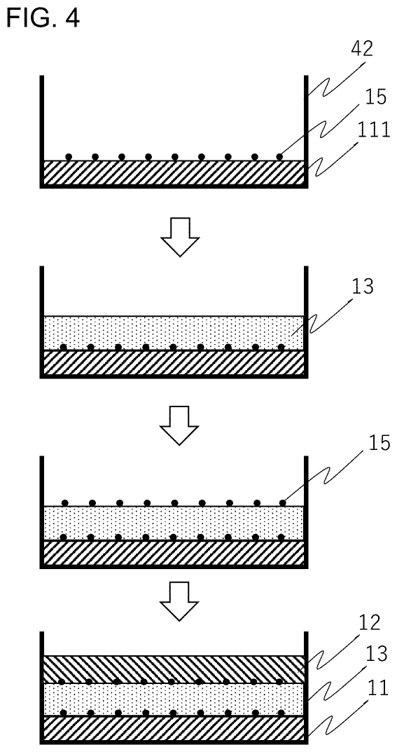

In addition, as another manufacturing method, as shown in FIG. 4, a gel or a resin composition constituting a first layer (or a second layer) is first cured by a chemical or physical operation to produce a gel sheet 111, and then the gel sheet 111 is arranged in a molding mold 42, and the markers 15 are arranged on an upper surface of the gel sheet 111. Next, a resin composition constituting an intermediate layer is injected into the molding mold 42 and gelled to form the intermediate layer 13. The markers 15 are arranged on an upper surface of the intermediate layer 13, and the resin composition constituting the second layer (or the first layer) is injected from above and cured. A timing of arranging the markers 15 on the upper surface of the intermediate layer 13 is preferably before the resin constituting the intermediate layer is completely gelled. In this method, an acoustic coupler in which the markers 15 are arranged (at the boundaries) between the intermediate layer 13 and the layers 11 and 12 on both sides of the intermediate layer 13 can be manufactured.

The method of FIG. 3 is suitable for a case where the marker is a wire, and since the markers are held inside the intermediate layer, the markers do not move even when the gel constituting the second layer is deformed, and a reliability of the indexes of the positions is high. In the method of FIG. 4, since the markers are arranged on a plane, a degree of freedom of the form of the markers is high, and since gel layers are sequentially laminated, the manufacturing method is also easy.

However, FIGS. 3 and 4 are merely examples of the method for manufacturing the acoustic coupler, and the method for manufacturing the acoustic coupler of the invention is not limited to these manufacturing methods. In addition, in FIGS. 3 and 4, each of the first layer 11 and the second layer 12 has a planar surface, but the shape of the surface is not necessarily planar, and may be a curved surface such as a spherical surface.

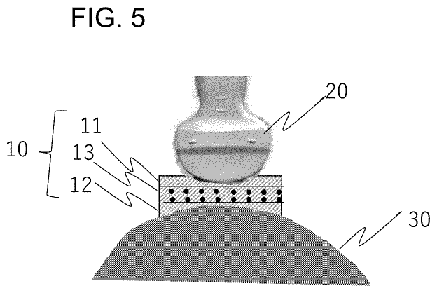

As shown in FIG. 5, in the acoustic coupler according to the present embodiment, the second layer 12 of the acoustic coupler 10 is placed on a subject 30 so as to be in contact with a body surface of the subject 30 and an ultrasound probe 20 is pressed against the first layer 11 side to perform imaging, when the ultrasound probe is in contact with the surface of the subject to perform the imaging. At this time, since the first layer 11 is formed by a gel having a high deformability, the acoustic coupler can be brought into contact with the body surface of the subject, which has unevenness or is the curved surface due to the deformation of the gel, without any gap.

In addition, since the intermediate layer 13 is formed by a hard gel having a high elastic modulus, even when the ultrasound probe 20 is pressed against the acoustic coupler 10, a pressing force is absorbed by the deformation of the first layer 11 and the second layer 12, and a flat shape of the intermediate layer 13 is maintained. Therefore, the arrangement of the markers 15 held in the intermediate layer is suppressed from being distorted, and a positional relationship between the ultrasound probe and the markers is maintained. When the ultrasound imaging is performed in this state, images of the markers 15 are superimposed on the ultrasound image by the ultrasound imaging device receiving the reflected waves from the markers 15, and a posture of the ultrasound probe, that is, the position (angle in a plane) and the inclination (angle with respect to the plane) of the ultrasound probe can be confirmed based on the images of the markers 15.

Next, a method for detecting the position (posture) of the ultrasound probe using the markers 15 in the ultrasound imaging using the acoustic coupler 10 having the above configuration will be described. Here, a case where the markers 15 are a grid-like wire (mesh) will be described as an example.

In the ultrasound image, an irradiation range of ultrasound waves is in an imaging region, and the imaging surface is determined by the direction and spread of the ultrasound waves emitted from the ultrasound probe. The spread of the ultrasound waves is determined by an arrangement of a transducer in the ultrasound probe and a beamformer of the ultrasound imaging device, and the irradiation direction is determined by the posture of the ultrasound probe. Therefore, if the posture of the ultrasound probe is known, a position of the imaging surface obtained at that time can be known. That is, as the posture of the ultrasound probe, when the surface of the subject with which the ultrasound probe is brought into contact is set as a reference surface, the irradiation region is determined by detecting the position and angle of the ultrasound probe in the reference surface and the inclination of the ultrasound probe with respect to the reference surface. When imaging is performed via the acoustic coupler, the reference surface is defined by a principal plane of the acoustic coupler.

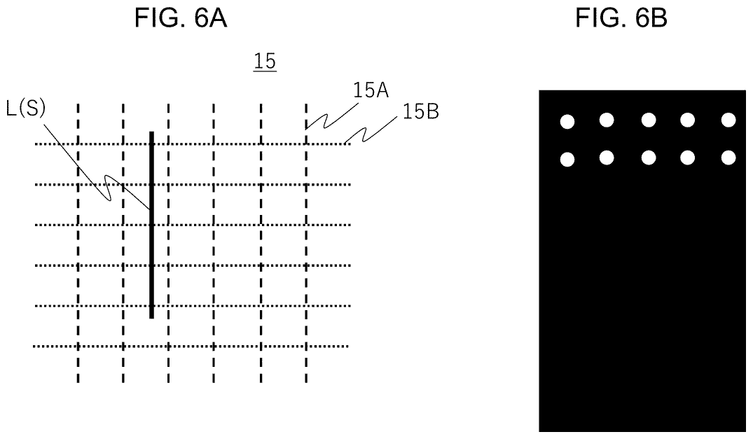

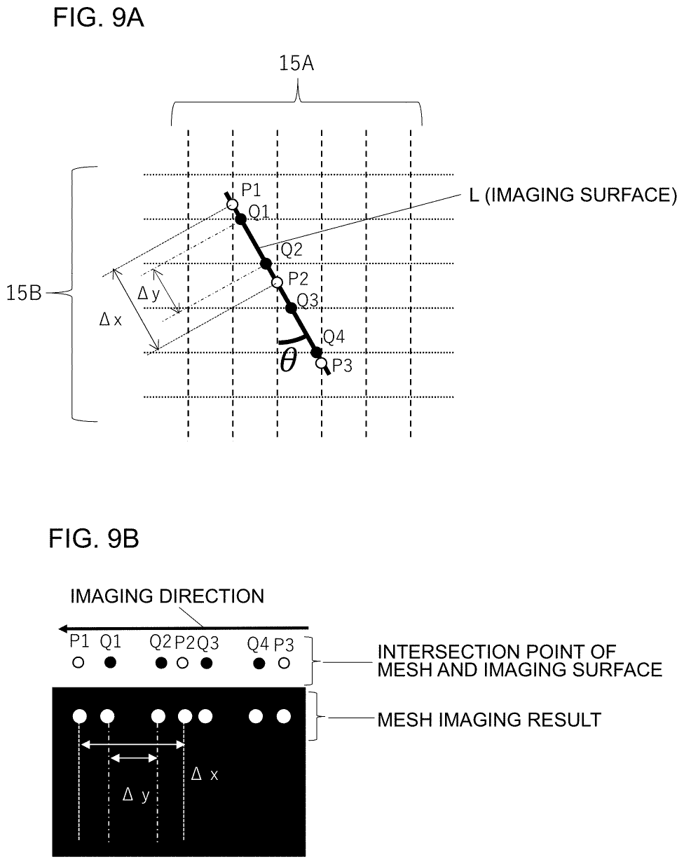

First, a case where a long axis direction of the ultrasound probe 20 is parallel to warps 15A or wefts 15B of the mesh will be described with reference to FIG. 6. FIG. 6A is a diagram of the reference surface viewed from above, and the long axis direction of the ultrasound probe is shown by a line L. FIG. 6B shows images of the markers when the irradiation direction of the ultrasound waves is perpendicular to the reference surface. That is, in FIG. 6A, an imaging surface (S) includes the line L and is a surface perpendicular to a paper surface. In FIGS. 9, 11, and 12 used in the description later, similarly to FIG. 6, each figure with A is a diagram of the markers (mesh) from above, and each figure with B shows an imaging surface.

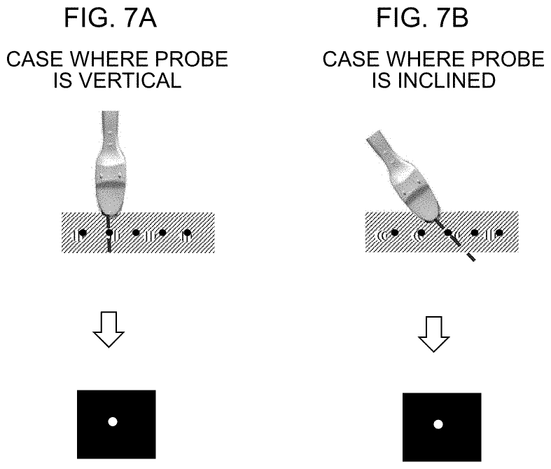

As shown in FIG. 6A, for example, when the ultrasound probe is parallel to the warps 15A of the mesh and does not overlap with the warps 15A, the imaging surface intersects with the wefts 15B of the mesh arranged above and below, and in the ultrasound image, as shown in FIG. 6B, the wefts 15B appear as images arranged at equal intervals. Here, as shown in FIG. 7, if the markers 15 exist on only one surface of the acoustic coupler, the same point images appear in both images of a case (a) where the imaging surface (irradiation direction of ultrasound waves) is perpendicular to the reference surface and a case (b) where the imaging surface is inclined and receives the reflected waves from the adjacent markers, so that both cases cannot be distinguished from each other.

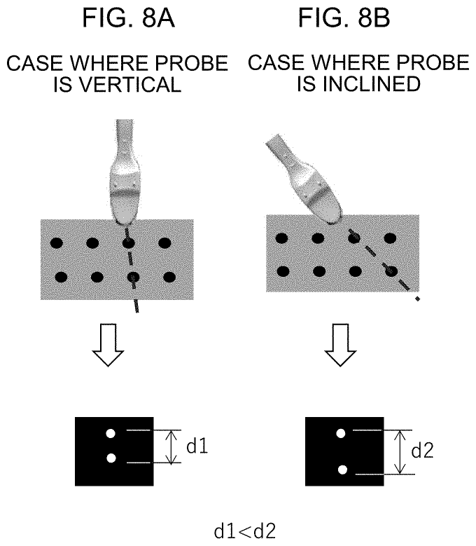

In response to this, when the markers are arranged on two surfaces, a distance between the point images of the first layer and the second layer increases due to a deep depth of the markers located far from an ultrasound source, and as shown in FIG. 8, both cases can be distinguished from each other based on a difference between a distance d1 in the case (a) where the imaging surface is perpendicular to the reference surface and a distance d2 in the case (b) where the imaging surface is inclined.

Next, when a long axis direction L of the ultrasound probe has a predetermined angle with respect to a longitudinal direction or a lateral direction of the mesh, as shown in FIG. 9A, the imaging surface crosses the warps and the wefts of the mesh, and intersection points of the ultrasound beam (imaging surface), the warps and the wefts appear as point images of the warps and the wefts, respectively. Here, as shown in FIG. 9B, when an angle θ of the imaging surface is less than 90 degrees with respect to the warp, more point images of the wefts appear than the point images of the warps, and in this example, point images P1, P2, and P3 of the warps and point images Q1, Q2, Q3, and Q4 of the wefts appear in an arrangement of P1, Q1, Q2, P2, Q3, Q4, and P3. If the angle θ exceeds 90 degrees, this is the same except that a relationship between the warp and weft is reversed.

Then, assuming that an interval between the wefts is α, an interval between the point images P1 and P2 of the adjacent warps is Δx, and an interval between the point images Q1 and Q2 of the adjacent wefts is Δy, the following relationships are established.

Δx=α/sin θ (1)

Δy=α/cos θ (2)

The interval α is a constant determined by the mesh and is constant assuming that the mesh does not deform.

Therefore, by obtaining Δx and Δy from the image, the angle θ of the ultrasound probe in the long axis direction can be known based on the following equation (3).

θ=tan⁻¹(Δy/Δx) (3)

Here, in order to obtain Δx and Δy from the image, it is necessary to distinguish between the point images of the wefts and the point images of the warps. There are several possible methods for distinguishing, and as one of the methods, there is a method of calculating a distance between the point images in a round-robin manner and using its appearance frequency (histogram). In the example of FIG. 9, there are a total of seven point images including three point images P1, P2, and P3 of the warps and four point images Q1, Q2, Q3, and Q4 of the wefts. Distances between P1 and the other six point images are calculated, distances between P2 and the other five point images are calculated, and so on, a total of 21 distances are calculated. Among the calculated distances, the interval Δy between the point images of the adjacent wefts and the interval Δx between the point images of the adjacent warps to be obtained are constant and appear at a predetermined frequency, but the appearance frequency of the distances between the point images of the warps and the point images of the wefts is low. Therefore, for example, the distance having the highest appearance frequency is Δx (or Δy), the point images used for calculating the distance are excluded, and the distance Δy (or Δx) is obtained from the remaining point images. Whether to set Δy or Δx can be determined, for example, by substituting the obtained distance into the equation (1) or the equation (2) to calculate θ and judging the value of θ is reasonable or not.

For example, when the angle θ is small, the appearance frequency of the interval Δy between the point images of the adjacent wefts is high, and the appearance frequency of the interval Δx between the point images of the adjacent warps is low. The relationship is reversed as the angle approaches 90 degrees. If the angle θ is calculated by the equation (1) using the interval Δx between the point images of the adjacent warps, and a is calculated by substituting the calculated angle and an interval Δy between the wefts into the equation (2), the value (calculated α) greatly deviates from the original α, and therefore, it can be seen that the distance having the highest appearance frequency is Δy instead of Δx.

In addition, as a different method, a method can be considered in which auxiliary markers for distinction are arranged only in one of a longitudinal direction and a lateral direction, and the longitudinal direction and the lateral direction are distinguished from each other by a difference in a pattern on a display of the marker when a portion of the probe is changed after the marker is attached to a measurement target. For example, as a preparation mode before measurement, when an operator performs an operation of moving the probe in a specific direction and the auxiliary markers arranged in the longitudinal direction are continuously seen, it can be distinguished that the markers are arranged in the longitudinal direction. As the auxiliary markers, it is considered that an image generated by overlapping markers having an arrangement interval different from that of other markers, or the markers on the ultrasound image is used.

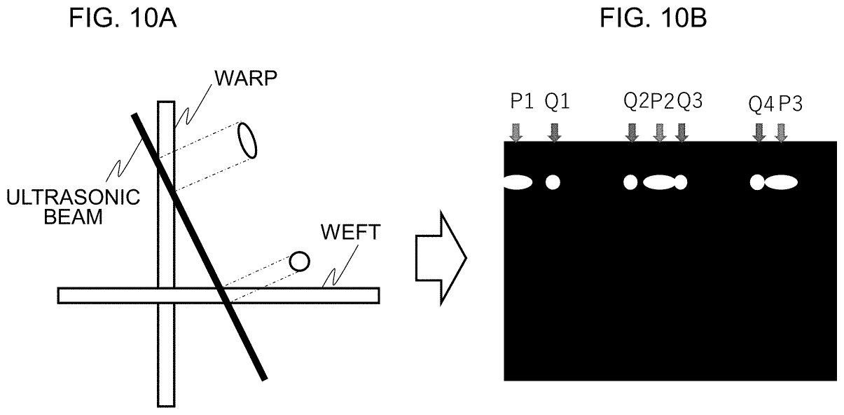

Further, as another method for distinguishing the warp and the weft, it is possible to distinguish the warp and the weft by utilizing the fact that an area of the ultrasound beams irradiated to the warp and the weft differs depending on the angle. For example, as shown in FIG. 10A, when the angle between the long axis direction of the ultrasound probe and the warp is small, the ultrasound beam that hits the warp is irradiated so as to diagonally cross the warp, and therefore the point image has a large eccentricity and a shape close to an ellipse. On the other hand, the ultrasound beam that hits the weft is irradiated at an angle close to perpendicular to the weft, so that the shape is close to a circle. The warp and weft are distinguished on the image based on a difference in the shape. In an example of FIG. 9, point images of the deformed warps 15A appear as shown in FIG. 10B, and the interval h between the adjacent wefts and an interval w between the adjacent warps can be measured. For an elliptical point image, a center position is a position of the point image.

This method is effective when there is a relatively large difference in irradiation angles of the ultrasound beam between the warp and the weft. The warp and weft may be distinguished by combining the two methods described above.

Next, a case where the ultrasound probe moves in parallel will be described.

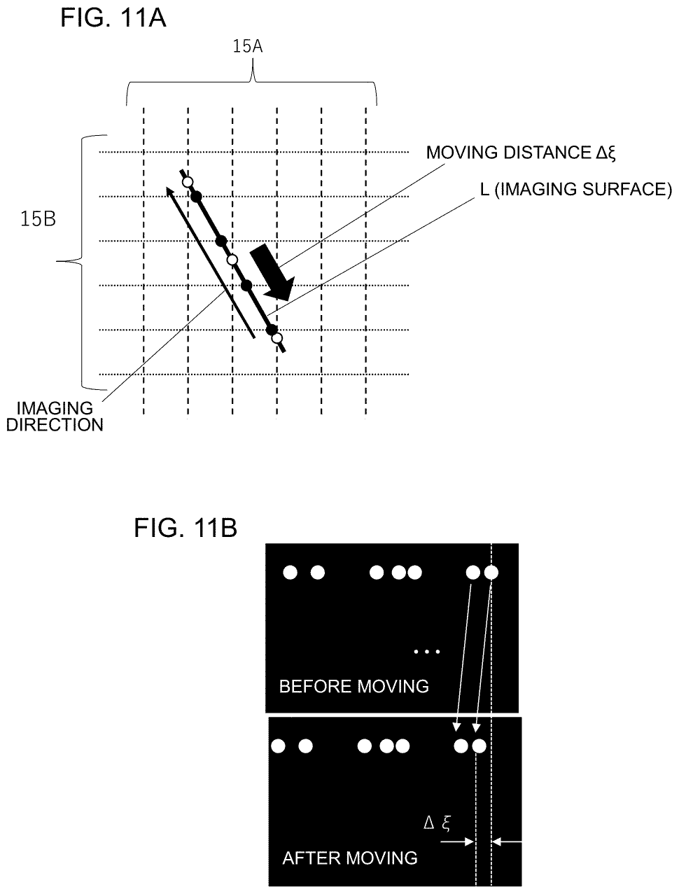

First, when the ultrasound probe moves in the long axis direction, all the point images move in the same direction while maintaining an arrangement relationship. FIGS. 11A and 11B show a state where the ultrasound probe has moved from the state of FIG. 9 and a change in the point images in the case. As shown in FIG. 11B, the point images move to a left side in this example by a shift amount Δξ corresponding to an actual movement amount of the ultrasound probe. By calculating the shift amount Δξ of the point images using a method such as optical flow, it is possible to know the movement amount of the ultrasound probe in the long axis direction.

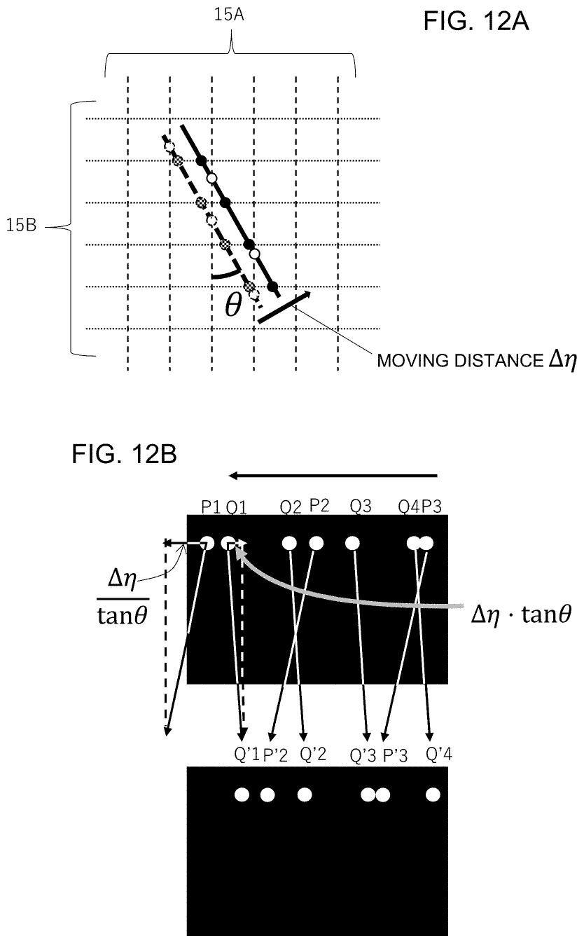

On the other hand, when the ultrasound probe moves in the short axis direction, shift directions of the point image of the warp and the point image of the weft are opposite as shown in FIGS. 12A and 12B. That is, the point images Q1, Q2, . . . of the wefts are shifted to a right direction in the figure on the image, but the point images P1, P2 . . . of the warps are shifted to a left direction in the figure, respectively. A shift amount Sp of the point image of the warp and a shift amount Sq of the point image of the weft are respectively expressed by the following equations using the angle θ in the long axis direction with respect to the reference surface described above, where the movement amount in the short axis direction is set as Δη.

Sp=Δη/tan θ

Sq=Δη·tan θ

Therefore, if the angle θ is known based on the equation (3), the movement amount Δη when the ultrasound probe is moved in the short axis direction at the angle θ can be calculated using the shift amount Sp and the shift amount Sq of the warp and the weft, respectively. In this case, the warp and the weft can be distinguished from each other by using a method using the above distance histogram.

As can be seen from the above equation, since the movement amount Δη is calculated using “tan θ”, the movement amount Δη cannot be calculated when θ is a multiple of 90 degrees, that is, when the long axis direction of the ultrasound probe is parallel to a direction of the warp or the weft, but by arranging the mesh on a lower side of the acoustic coupler 10 (on a deeper side with respect to the ultrasound wave propagation direction) so as to be shifted by 45 degrees with respect to the mesh on an upper side of the acoustic coupler 10, it is possible to calculate the movement amount Δη using the shift amount of the point images of the mesh on the lower side (the warp and the weft).

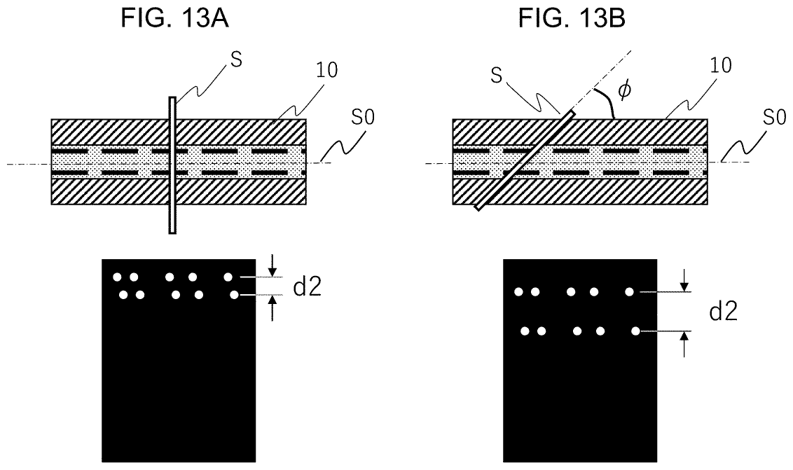

Next, a detection of inclination φ when the imaging surface S is inclined from a surface perpendicular to a reference surface S0 will be described. When the imaging surface S is perpendicular to the reference surface S0, the distance between the point images of the meshes arranged above and below (the upper and lower layers in a line direction) is the shortest (FIG. 13A), but when the imaging surface S is inclined with respect to the reference surface S0, the distance between the first layer and the second layer becomes longer as shown in FIG. 13B. That is, assuming that the distance between the layers in the case where the imaging surface is perpendicular to the reference surface is d1 and the distance between the layers in the case where the imaging surface is inclined with respect to the reference surface is d2, the inclination φ is expressed as d2 cos φ=d1. Since the distance d1 between the layers is determined by a structure of the acoustic coupler, if d2 is known, the inclination φ can be calculated.

As described above, by using the acoustic coupler in which the markers arranged in the two-dimensional direction are arranged in the two layers having different positions in a thickness direction, respectively, a rotation of the imaging surface in the reference surface (the angle θ with respect to a reference direction), the movement amounts (Δξ, Δη) in the long axis direction and the short axis direction, and the inclination (φ) with respect to the reference surface can be detected by analyzing the images (point images) of the markers.

The case where the markers are the mesh formed by the warps and the wefts has been described above, but the posture of the ultrasound probe can be detected in the same manner for the particles and the bubbles in which the markers are two-dimensionally arranged.

Next, a configuration of the ultrasound imaging device corresponding to the acoustic coupler according to the present embodiment will be described.

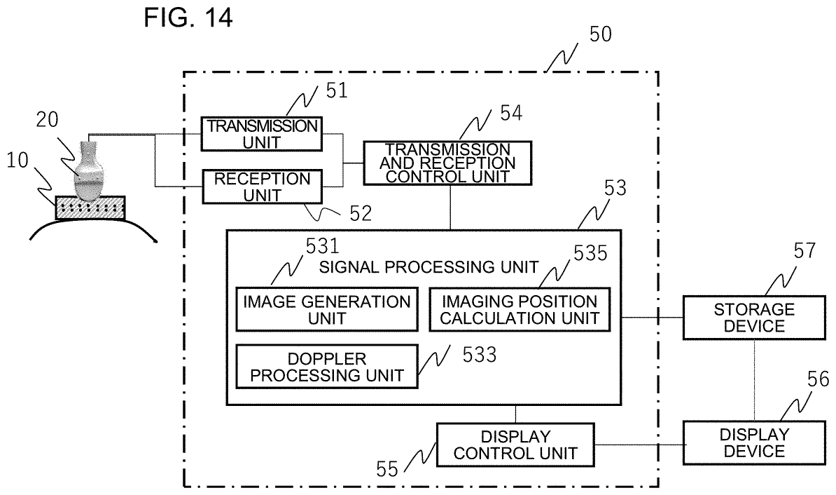

FIG. 14 is a diagram showing an overall configuration of the ultrasound imaging device, and similarly to a well-known ultrasound imaging device, an ultrasound imaging device 50 includes a transmission unit 51 that transmits an ultrasound signal to the ultrasound probe 20, a reception unit 52 that receives an echo signal from an imaging target detected by the ultrasound probe 20, a signal processing unit 53 that processes the signal received by the reception unit 52 to generate an ultrasound image, a transmission and reception control unit 54 that controls transmission and reception, a display control unit 55 that generates a display image to be displayed on a display device using the ultrasound image generated by the signal processing unit 53, and the like. In addition, an accessory device of the ultrasound imaging device 50 may include a display device 56 that displays the ultrasound image or the like and a storage device 57 that stores a processing result of the signal processing unit 53 such as the ultrasound image.

The transmission unit 51 and the reception unit 52 include a beamformer that adjusts a phase of the ultrasound wave in accordance with the imaging target, and transmit the ultrasound signal to the ultrasound probe 20 and transmit the echo signal from the ultrasound probe to the signal processing unit 53 as a signal for each frame, for example, in accordance with the ultrasound image and Doppler information to be acquired under a control of the transmission and reception control unit 54.

Similarly to a normal ultrasound imaging device, the signal processing unit 53 includes an image generation unit 531 that generates a B-mode image or the like, a Doppler processing unit 533 that calculates blood flow information based on the echo signal, and the like, and further includes an imaging position calculation unit 535 that calculates the position information (the movement amount, the angle, and the inclination) of the ultrasound probe 20 using the image (B-mode image) generated by the image generation unit 531.

When the imaging position calculation unit 535 receives the ultrasound image created by the image generation unit 531, the imaging position calculation unit 535 specifies the images (point images) of the markers included in the image and performs various calculations for determining the imaging surface of the ultrasound waves. Specifically, the imaging position calculation unit 535 performs various calculations such as calculation of the distance between the point images, creation of the distance histogram, determination of the shape of the point images, and calculation of a movement amount of a point image group (calculation of the optical flow).

As one function of the signal processing unit 53, such a function of the imaging position calculation unit 535 may be implemented by software by a computer including a CPU, a GPU, and a memory, or may be implemented by hardware such as an ASIC or an FPGA. In addition, the function may be implemented by a computer or hardware separate from the signal processing unit 53.

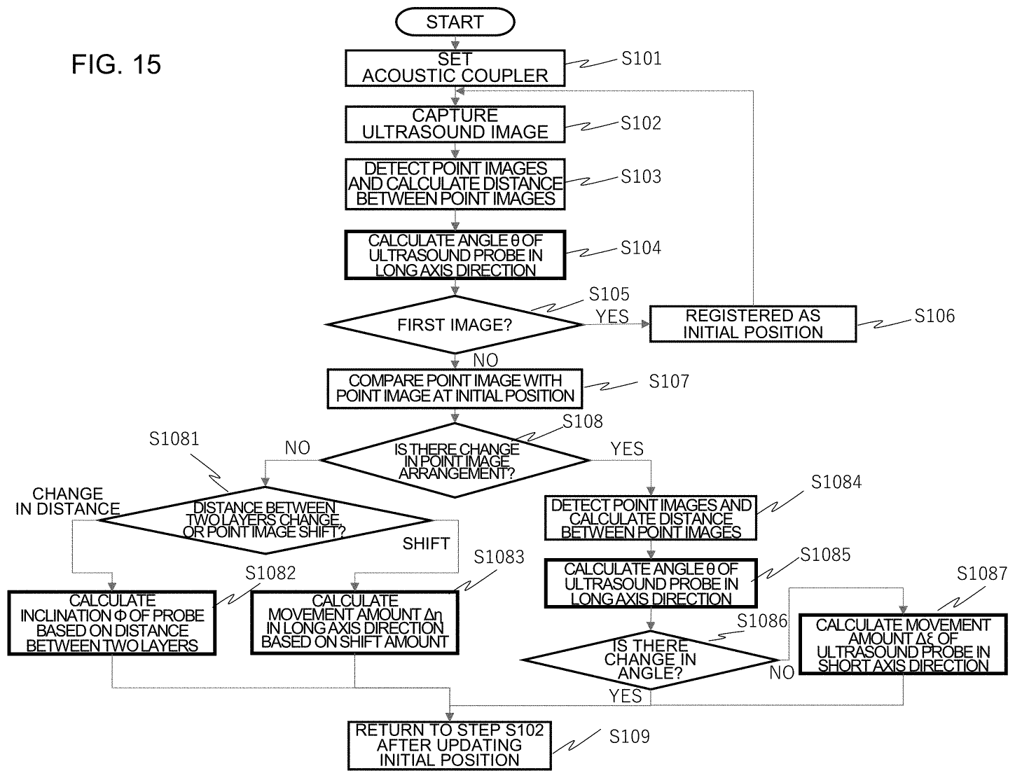

FIG. 15 shows an example of a procedure for calculating the position information of the ultrasound probe 20 by the imaging position calculation unit 535.

When an imaging using the acoustic coupler of the invention is set (S101), the imaging position calculation unit 535 operates and starts capturing an image from the image generation unit 531 (S102). Whether the imaging using the acoustic coupler of the invention is performed may be set, for example, by a user via an input device (not shown), or imaging using the acoustic coupler may be set by default, and it may be automatically determined that the acoustic coupler of the invention is not used when there is no marker in the image.

When a first ultrasound image is captured, the imaging position calculation unit 535 detects the positions of the markers from the point images included in the first ultrasound image, detects the distance between the point images, and creates the distance histogram (S103). The imaging position calculation unit 535 calculates the angle θ of the ultrasound probe in the long axis direction from the histogram (S104). For example, when the long axis direction of the ultrasound probe is parallel or substantially parallel to the warp or the weft of the mesh, the distance between the point images is constant, and only that distance appears in a peak shape in the histogram. When a plurality of distances appear in the histogram, the distance between the point images of the adjacent warps and the distance between the point images of the adjacent wefts are determined based on the frequency, and the angle θ is calculated. The position and the angle calculated in step S104 are stored as an initial position of the first ultrasound image (S105, S106).

Next, each time an image is captured while the ultrasound probe is moved, the point images of the markers of the obtained image are detected and compared with an arrangement of initial point images, and it is determined whether there is a movement in the long axis direction, a movement in the short axis direction, a change in the angle, or a change in the inclination (S107). That is, when there is no change in an arrangement pattern of the point images (S108), it is determined whether the interval between the point image of the first layer and the point image of the second layer has changed or the pattern of the point image has shifted in a lateral direction (azimuth direction) of the image (S1081). Compared with the interval at the initial position between the first layer and the second layer, when the interval changes (FIG. 13), it is determined that the inclination φ of the ultrasound probe has changed, and the inclination of the ultrasound probe is calculated based on that distance (S1082). After that, the inclination at the initial position is updated, and the process returns to step S102 (S109).

When the point images are shifted in the lateral direction (azimuth direction) of the image while the point images maintain the arrangement pattern (S1082), it is determined that there is the movement in the long axis direction (FIG. 11), and the movement amount in the long axis direction is calculated based on the shift amount Δη of the point images (S1083). After that, the positions in the long axis direction at the initial position are updated, and the process returns to step S102 (S109).

In addition, when it is determined in step S108 that the distance between the point images has changed, it is determined that movement or rotation (change in the angle) in the short axis direction has been performed. First, similarly to steps S103 and S104, the distance between the point images is calculated in a round-robin manner, the histogram is created, and the angle θ is calculated (S1084 and S1085). If the angle θ is the same as the angle θ obtained in step S104 (S1086), it can be regarded as a movement in the short axis direction (FIG. 12), and therefore the movement amount in the short axis direction is calculated using the shift amount of the point images of the warps and the shift amount of the point images of the wefts (S1087). After that, the positions in the short axis direction at the initial position are updated, and the process returns to step S102 (S109).

If the angle calculated in step S1085 is different from the angle registered as the initial position (S1086), the initial position is updated at the angle, and the process returns to step S102 (S109).

With respect to the initial position, the above steps are repeated while updating the initial position each time there is any change, and the position information of the imaging surface is acquired for each image.

The position information of the ultrasound probe 20 calculated by the imaging position calculation unit 535 is stored in the storage device 57 together with the ultrasound image used for the calculation. Alternatively, the position information and the ultrasound image are passed to the display control unit 55, and the display control unit 55 displays the position of the ultrasound probe when the ultrasound image is captured together with the ultrasound image on the display device 56.

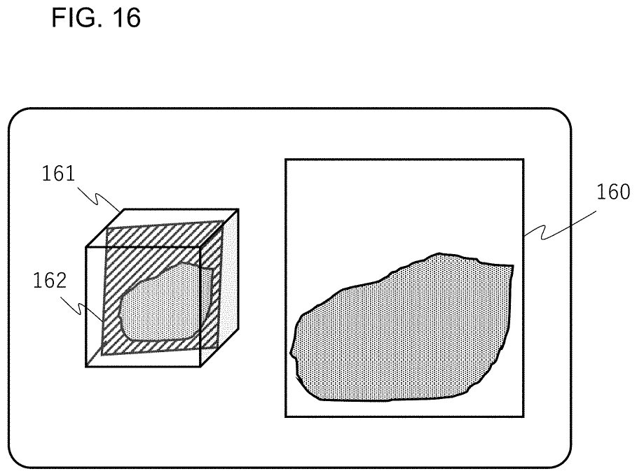

Although a manner of display the result is not particularly limited, for example, as shown in FIG. 16, an ultrasound image 160 which is a two-dimensional image and an image 161 indicating a three-dimensional imaging region may be displayed together, and an imaging surface 162 determined by the ultrasound probe may be displayed in the three-dimensional image. Therefore, the operator can confirm at a glance where the displayed ultrasound image is capturing an image of the subject, and can also confirm whether there is no omission of scanning or whether an order of scanning matches a preset procedure.

Such display may be performed in real time during imaging, or may be performed using the ultrasound image stored in the storage device 57.

According to the ultrasound imaging device according to the present embodiment, when imaging is performed using the acoustic coupler according to the present embodiment, the position information can be calculated and presented in response to the change in the position of the ultrasound probe. Therefore, the operator can screen an imaging range without causing omission, and can capture an image of the confirmed place again as necessary.

Although the case where the acoustic coupler 10 and the ultrasound probe 20 are separate bodies has been described in the above embodiment, the acoustic coupler 10 of the invention can be used by being fixed to the ultrasound probe 20.

What is claimed is:

1. An acoustic coupler, comprising:
a first layer in contact with a probe of an ultrasound imaging device; and
a second layer in contact with an imaging target, wherein
a plurality of markers are provided between the first layer and the second layer at different positions with respect to an ultrasound wave propagation direction.

2. The acoustic coupler according to claim 1, further comprising:
an intermediate layer having a higher elastic modulus than the first layer and the second layer, between the first layer and the second layer.

3. The acoustic coupler according to claim 2, wherein the plurality of markers are arranged between the first layer and the intermediate layer and between the second layer and the intermediate layer, respectively.

4. The acoustic coupler according to claim 2, wherein the plurality of markers are embedded in the intermediate layer.

5. The acoustic coupler according to claim 2, wherein the intermediate layer has an elastic modulus of 10 kPa or more in Young's modulus.

6. The acoustic coupler according to claim 1, wherein the plurality of markers are any of striatum, particles, and bubbles.

7. The acoustic coupler according to claim 6, wherein the markers are a grid-shaped striatum.

8. The acoustic coupler according to claim 1, wherein the acoustic coupler has a sheet shape.

9. An ultrasound image processing method for arranging an acoustic coupler between an inspection target and an ultrasound probe, transmitting and receiving an ultrasound wave to and from the inspection target via the ultrasound probe, and processing a generated ultrasound image of the inspection target, the ultrasound image processing method comprising:
using an acoustic coupler according to claim 1 as the acoustic coupler and specifying a plurality of markers of the acoustic coupler in the ultrasound image; and
calculating position information of the ultrasound probe using a positional relationship of the plurality of specified markers.

10. The ultrasound image processing method according to claim 9, comprising:
specifying a plurality of markers of the acoustic coupler in ultrasound images acquired at a first position and a second position of the ultrasound probe, respectively; and
calculating a moving direction and a moving angle of the ultrasound probe using positions of the markers in the ultrasound image at the first position and positions of the markers in the ultrasound image at the second position.

11. An ultrasound imaging device, comprising:
a transmission and reception unit to which an ultrasound probe is connected and configured to transmit and receive an ultrasound wave via the ultrasound probe;
an image generation unit configured to generate an ultrasound image using an ultrasound wave that is a reflected wave received from an inspection target; and
an imaging position calculation unit configured to calculate position information of the ultrasound probe based on a position of an image of a marker included in the ultrasound image, wherein the ultrasound image is an image captured by interposing the acoustic coupler according to claim 1 between the inspection target and the ultrasound probe.
